(19) United States
(12) Patent Application Publication
(10) Pub. No.: US 2014/0032234 A1
(43) Pub. Date: Jan. 30, 2014

(54) HEALTH AND WELLNESS SYSTEM

(71) Applicant: VaporWire, LLC, Greenfield, MN (US)

(72) Inventor: Brady C. Anderson, Greenfield, MN (US)

(73) Assignee: VaporWire, LLC, Greenfield, MN (US)

(21) Appl. No.: 13/952,319

(22) Filed: Jul. 26, 2013

Related U.S. Application Data
(60) Provisional application No. 61/676,160, filed on Jul. 26, 2012.

(51) Int. Cl. G06Q 50/22; A61B 5/00; A61B 5/0205; G01G 19/44; A61B 5/11; A61B 5/145

(52) U.S. Cl. CPC G06Q 50/22; A61B 5/112; A61B 5/14532; A61B 5/0205; G01G 19/44; A61B 5/0022
USPC 705/2; 600/595; 600/508; 600/485; 600/365; 600/483; 600/301; 177/1

(57) ABSTRACT

A health and wellness social network can collect digital health and wellness information about network users and incentivize the users to engage in a more healthy and active lifestyle. In one example, a method includes receiving, at a server device, data related to a health of a user, the data being generated by a health measurement device configured to transmit the data over a network to the server device. The techniques also includes, responsive to receiving the data related to the health of the user, determining, by the server device, a wellness reward for the user, where different wellness rewards are associated with different data received from the user. The technique also includes modifying, by the server device, a user profile associated with the user to reflect the wellness reward.

### CROSS-REFERENCE

This application claims the benefit of U.S. Provisional Patent Application No. 61/676,160, filed Jul. 26, 2012, the entire contents of which are incorporated herein by reference.

### TECHNICAL FIELD

This disclosure relates to systems for promoting the health and wellbeing of individuals and, in particular, to computing software for managing health data, incentivizing individuals to engage in healthy behavior, and networking individuals in a health community to increase compliance with healthy behaviors.

### BACKGROUND

With an aging population and ever increasing healthcare costs, individual health and wellbeing is a topic of increasing importance in modern society. Employers, insurers, and healthcare providers, among other parties, are increasingly taking an active interest in understanding and encouraging individuals to lead healthy, active lifestyles. Leading a healthy, active lifestyle can reduce healthcare costs associated with treating acute and chronic conditions, reduce the patient burden on the healthcare system, and improve the overall quality of life for the participating individual. Unfortunately, many individuals lack the time, motivation, or even understanding about what actions they should be undertaking in their day-to-day lives to improve their overall health and wellbeing.

Although individuals do not necessarily have the guidance and motivation necessary to improve their physical health and wellbeing, individuals are increasingly being influenced in other aspects of life through interaction with social networking communities. Social networking embraces the general concept that an individual's online personal network of friends, family, co-workers, employers, and other contacts can be utilized to find more relevant connections for dating, job networking, purchasing decisions, finding information, service referrals, and the like. The amount and way information is shared through social networking can be useful to help manage an individual's affairs and encourage the individual to change their behaviors through interaction with another individual or group of individuals. The power of social networking is often limited, however, because the virtual nature of the interaction does necessarily translate to incentives and information that promote real-world physical behavior changes.

### SUMMARY

In general, this disclosure is directed to a digital health and wellness platform for automatically collecting information about the health of a user and incentivizing the user to engage in a more healthy and active lifestyle. In some examples, the disclosed techniques involve a server that hosts multiple users linked together in a social health and wellness network. The users may all belong to the health and wellness network and have profiles that are associated with the network and accessible by the server. For example, within the network, users may belong to different communities (e.g., by associating their profiles with the communities), such as communities of friends, families, employers, or the like. Within each community, different incentives or rewards may be established to encourage the users to engage in conduct that is likely to improve their health and wellbeing.

To help monitor the behavior of users of the social health and wellness network, the users interact with health measurement devices to generate data about the health of the users. For example, the users may carry a device that measures their activity level, e.g., during the course of a typical day or during a defined period of activity. As another example, the users may interact with a device that measures their particular health state such as, e.g., a scale or blood pressure monitor. Data generated by one or more health measurement devices can transmit over a network to the server hosting the social health and wellness network. In some examples, the health measurement device(s) automatically and wirelessly transmit the data to the network so as to minimize barriers to user compliance with a monitoring program. Regardless, the server may associate the data with the user's profile (e.g., store the data with the user's profile) and analyze the data to evaluate the health and wellness of the user. A user interface associated with the user's profile for the social health and wellness network may update to reflect changes in the health and wellness of the user as determined from the data received from the health measurement device.

To incentivize the users to engage in activities that will improve their health and wellbeing, the social health and wellness applications and network offers incentive awards to the user, encouraging the user to engage in certain conduct. For example, other users of the social health and wellness network, an administrator of the social health and wellness network, or sponsors of the social health and wellness network (e.g., insurance companies, employers, fitness centers) can offer incentives for network users to engage in desirable conduct. The incentives can be physical items (e.g., shirts, gift certificates, physical fitness supplies), virtual items (e.g., increased status within the social network community, digital content such as copyrighted movies, books, music) or the like. The user can earn wellness rewards (e.g., points, grades) until they reach a wellness reward level (e.g., a certain number of points or a certain grade) that is sufficient to receive an incentive award. For example, the user can earn wellness rewards for engaging with the social health and wellness network (e.g., logging into the network, posting content, sending messages to other users in the network) and for performing physical activities (e.g., physical activity recorded by a health measurement device carried by the user and sent to the server). The physical activities may be general day-to-day physical activities (e.g., walking, running, biking, climbing stairs) or physical activities specifically identified by the social health and wellness network by way of a physical fitness challenge or goal for the user to complete. Different magnitudes of wellness rewards can be associated with different activities.

Upon accumulating enough wellness rewards to earn an incentive award, the server may issue a notification to the user that the user has earned the incentive award. In some examples, the server issues the notification in substantially real-time with the user earning the incentive reward. The server may receive data from the health measurement device carried by the user as the user is performing a physical activity and determine a wellness reward (e.g., a certain number of points associated with the activity). The magnitude of the wellness reward may increase as the user continues performing the physical activity. Accordingly, the server may determine in substantially real-time when the user has accumulated enough wellness rewards to earn the incentive award and issue a notification in substantially real-time to the user informing them of such fact. For example, the server may transmit a text message, an e-mail message, or a mobile application alert to a mobile electronic device carried by the user. Depending on the circumstances, the user may receive the notification of earning the incentive award via their mobile device while still performing the physical activity that caused the user to accumulate enough wellness rewards to earn the incentive award. Unlike an incentive award that a user learns about days or weeks after earning, the substantially real-time notification may reinforce the positive behavior of the user and cause the user to continue their immediate physical activity. This may promote long-term positive behavioral changes.

In addition to or in lieu of incentive awards, the social health and wellness network may transmit messages to users encouraging them to stop, begin, or continue conduct, e.g., conduct that has been clinically shown to promote a healthy lifestyle. For example, a user of the social health and wellness network may transmit a message of encouragement to another user within a shared community of the network. As another example, the server hosting the network may analyze conduct of a user (e.g., based on data received from a health measurement device used by the user) and send a message to the user encouraging the user to stop certain conduct, begin certain conduct, or continue performing certain conduct. In different examples, the message may be in the form of a post to a user interface associated with the user's profile on the network, an e-mail message to the user's e-mail account, a text message to a portable device of the user, an application alert on the portable device of the user, or the like.

The social health and wellness network may function as a platform for providing a variety of other user engagement and user monitoring functions so as to encourage the user to engage in a healthy lifestyle. For example, the social health and wellness network may function to monitor and validate user behavior for distribution of monetary funds associated with the behavior. One example of such a monetary fund is a certain amount of money made available to a user by an employer, insurer, or other party that the user can only receive if the user engages in a certain type of predefined behavior. Another example of such a monetary fund is a certain amount of money that is authorized by government regulation to be placed in a tax-free account (e.g., similar to a health savings account) but which the user can only withdraw tax-free if he engages in a certain type of predefined behavior. The predefined behavior may be a user engaging in a certain magnitude, type, and/or duration of activity, and/or a user checking their health state with a certain frequency. The social health and wellness network may automatically receive data from networked health measurement devices used by the user and determine if the user has met the requirements for receiving the monetary funds. If so, a server associated with the social health and wellness network may issue forms, an authorization code, or otherwise provide access for the user to access the monetary funds.

In one example, a method is described that includes receiving, at a server device, data related to a health of a user, the data being generated by a health measurement device configured to transmit the data over a network to the server device and responsive to receiving the data related to the health of the user, determining, by the server device, a wellness reward for the user. According to the example, different wellness rewards are associated with different data received from the user. The example method further includes modifying, by the server device, a user profile associated with the user to reflect the wellness reward.

In another example, a non-transitory computer readable medium is described that includes instructions that cause a processor to receive data related to a health of a user, the data being generated by a health measurement device configured to transmit the data over a network, and responsive to receiving the data related to the health of the user, determine a wellness reward for the user, where different wellness rewards are associated with different data received from the user. The non-transitory computer readable medium also includes instructions that cause the processor to modify a user profile associated with the user to reflect the wellness reward.

In another example, a system is described that includes an interface of a server device that receives data related to a health of a user, the data being generated by a health measurement device configured to transmit the data over a network to the interface of the server device, a wellness rewards module that determines a wellness reward for the user based on the received data, and a user interface control module that modifies a user profile associated with the user to reflect the wellness reward.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a block diagram illustrating an example network-based environment for providing health and wellness monitoring and encouragement.

FIG. 2 is a block diagram illustrating an example arrangement of components of an example server that may be used in the network-based environment of FIG. 1.

FIG. 3 is screen diagram illustrating an example user interface that may be presented to a user in accordance with one aspect of this disclosure.

FIG. 4 is screen diagram illustrating another example user interface that may be presented to a user in accordance with one aspect of this disclosure.

FIG. 5 is screen diagram illustrating another example user interface that may be presented to a user in accordance with one aspect of this disclosure.

### DETAILED DESCRIPTION

In general, this disclosure is directed to devices, systems, and techniques for monitoring the health and welfare of an individual and encouraging the individual to engage in conduct that improves their health and wellbeing. As described in some examples herein, the techniques involve a social network system that supports a social network with multiple users. Users may be organized into different groups, where the amount of information available about a user and/or the ability to communicate with a particular user may depend on whether or not an individual is in a group with the user. The techniques may also involve one or more health measurement devices that communicate with a computing device supporting the social network system. For example, data measured by the health measurement device may indicate the type, magnitude, and/or duration of different physical activities engaged in by the user over a time period in which the user is carrying the device. As another example, data measured by the health measurement device may indicate a health state of the user such as, e.g., body weight, blood pressure, or the like.

A computing device supporting the social network may store a user profile associated with each user of the social network. The computer device may present a user interface for each user, e.g., accessible through a web-based interface or mobile application, where information about the user is available to other users within the social network. For example, the user interface may present information about physical activity the user has engaged in over a certain period of time (e.g., the last hour, day, week, or month) and/or various health states of the user (e.g., weight, blood sugar). The user interface may also display health and wellness goals established by or for the user and the user's progress towards those goals. For example, the user interface may display an incentive reward the user is working toward earning, a goal weight the user is working to achieve, a certain amount of physical exercise the user desires to engage in over a given period of time, or the like. By identifying their health and wellness goals and progress towards those goals to other members within the social network, a user's motivation to actually achieve the goals will be greater than if the user's health and wellness goals are kept private. For example, the social influence of other network members encouraging a user to accomplish their goals and/or the adverse social influence associated with not meeting a public goal can help incentivize a user to engage in conduct that will achieve the goal. Further, other members of the network and/or the server providing the network itself can offer guidance to help the user identify goals and behavior changes that will lead to a healthier lifestyle. The social health and wellbeing network can provide a number of other functions, as described herein.

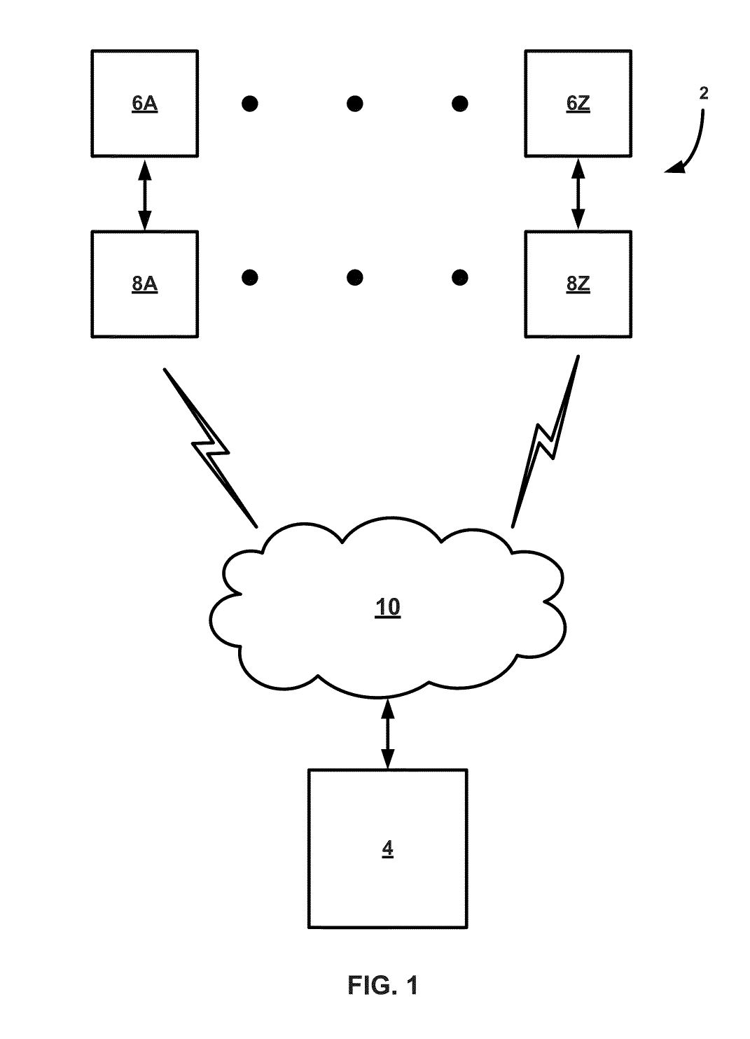

FIG. 1 is a block diagram illustrating an example network-based environment 2 in which a social health and wellness network server 4 provides network-based health and wellness monitoring and encouragement. As described herein, social health and wellness network server 4 provides a user interface with which users 6A-6Z (collectively “users 6”) interact to perform a wide range of tasks associated with monitoring and encouraging an individual's health and wellness. A user may be, for example, an individual, a family member, an employer, an employee, an insurer, a healthcare provider, a social network host administrator, or any other user potentially interested in an individual's health and wellness. As shown, user 6 communicates with social network server 4 via user devices 8A-8Z (collectively “user device 8”) over a network 10. Network 10 is in communication with and enables communication between user devices 8 and social network server 4.

User devices 8 may include virtually any computing device capable of receiving and sending information over a network, such as network 10, to and from another computing device, such as social network server 4, each other, and the like. User devices 8 may include devices that typically connect using a wired communications medium such as personal computers, multiprocessor systems, microprocessor-based or programmable consumer electronics, network PCs, and the like. User devices 8 may also include devices that typically connect using a wireless communications medium such as cell phones, smart phones, tablet computers, pagers, radio frequency (RF) devices, infrared (IR) devices, integrated devices combining one or more of the preceding devices, or virtually any mobile device, and the like. In some examples, user devices 8 may be any device that is capable of connecting using a wired or wireless communication medium such as a PDA, wearable computer, and any other device that is equipped to communicate over a wired and/or wireless communication medium.

Each user device within user devices 8 may include an application (e.g., a browser application) that is configured to receive and to send data to social network server 4. The application may be configured to receive and display graphics, text, multimedia, and the like. User devices 8 may be further configured to receive a message from another computing device employing another mechanism, including, but not limited to email, Short Message Service (SMS), Multimedia Message Service (MMS), instant messaging (IM), internet relay chat (IRC), and the like.

User devices 8 may be further configured to enable a user to manage a user profile, view and manage health and wellness information, activity participation, and the like, which may in turn be stored on a user's device and/or at a remote location, such as social network server 4. Accordingly, user devices 8 may further include a client application that is configured to manage various actions on behalf of the client device. For example, the client application may enable a user to interact with a browser application, email application, and the like, to customize how another social network user might view a profile associated with the user. For example, the user may employ the client application, in part, to provide one customized view for family members, another customized view for co-workers, and the like.

To facilitate generation of health and wellness data for users 6 during operation of network environment 2, user devices 8 may be configured as health measurement devices that make measurements related to the health of users 6. For example, user devices 8 may make measurements concerning the type, magnitude, and/or duration of different physical activities engaged in by user 6 over the time period in which a user is carrying the device. In such examples, each user device may be implemented as a pedometer, heart rate monitor, or other device configured to measure biometric data of a user. Data generated by each user device may be indicative of an activity level of a user which, in turn, can be used as a proxy for the health and wellness of the user. The data may be stored on a user device and/or at a remote location, such as social network server 4.

As another example, user devices 8 may make measurements concerning a health state of user 6. A health state of a user may be a parameter (e.g., a physiological parameter) that indicates the health of the user but that does not necessarily vary based on the activity level of the user. Example health states of the user may include weight, height, blood pressure, blood glucose level, white blood cell count, and the like. In such examples, each user device may be implemented as a scale, blood pressure monitor, blood glucose monitor, or the like. Such devices may automatically communicate data concerning a health state of a user as the data is generated by the device. The data may be stored on a user device and/or at a remote location, such as social network server 4.

In some examples, a single user may be associated with multiple user devices 8. For example, a user may be associated with a first user device that enables the user to control interaction with social network server 4, receive textual or voice messages, or the like. The first user device may communicate over a wireless communications medium such as a cell phone, smart phone, or tablet computer. The user may also be associated with a second user device that is configured as a health measurement device. The health measurement device may be carried by the user (e.g., worn by the user) to measure activity levels, health conditions, or other health and wellness parameters of the user. The health measurement device may communicate over a wireless communications medium, e.g., continuously or at defined intervals to provide data monitored and/or determined by the health measurement device to network server 10.

Network 10 is configured to couple one computing device to another computing device to enable the devices to communicate together. Network 10 is enabled to employ any form of computer readable media for communicating information from one electronic device to another. Also, network 10 may include a wireless interface, and/or a wired interface, such as the Internet, in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router may act as a link between LANs, enabling messages to be sent from one to another. Communication links within LANs may include twisted wire pair or coaxial cable, while communication links between networks may utilize analog telephone lines, full or fractional dedicated digital lines, Integrated Services Digital Networks (ISDNs), Digital Subscriber Lines (DSLs), wireless links including cellular and satellite links, or other communications links. Furthermore, remote computers and other related electronic devices may be remotely connected to either LANs or WANs via a modem and temporary telephone link.

The media used to transmit information in communication links as described above illustrates one type of computer-readable media, namely communication media. Generally, computer-readable media includes any media that can be accessed by a computing device. Computer-readable media may include computer storage media (e.g., non-transitory computer readable storage media), communication media, or any combination thereof.

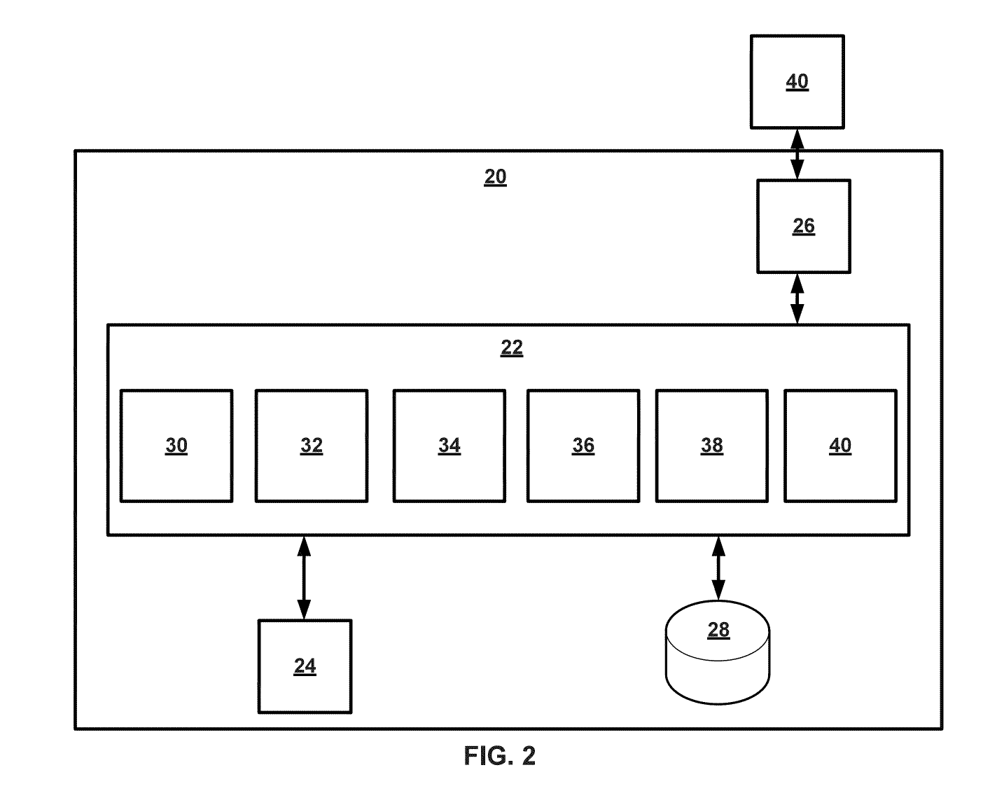

One example configuration of social network server 4 is described in more detail below with respect to FIG. 2. Briefly, however, social network server 4 may include any computing device or combination of computing devices capable of connecting to network 10 to receive data from user devices 8, transmit information to user devices 8, or otherwise control monitoring and encouragement activity to promote users 6 to lead more healthy and active lifestyles. Devices that may operate as social network server 4 include personal computers, desktop computers, multiprocessor systems, microprocessor-based or programmable consumer electronics, network PCs, servers, and the like.

Social network server 4 may be configured to receive information associated with a user (e.g., monitored health data of a user) and analyze the data to determine an activity level of the user, wellness rewards earned by the user, incentive awards earned by the user, and the like. Social network server 4 may also be configured to transmit a message of encouragement to a user to encourage the user to stop, start, and/or continue certain behavior. In addition, social network server 4 may initiate wellness challenges to incentivize users to engage in a challenge activity beneficial to their health and wellbeing. Wellness rewards can be earned upon completing a wellness challenge.

Social network server 4 may store a user profile created by or for each user of users 6. User information associated with each user may include any information about the respective user that the user desires to make accessible to other users of the social network. For example, user information may include a name and/or an alias, a representative image (e.g., a photographic image), contact information, health status information (e.g., weight, current activity level), a list of health and wellness goals, and/or other information regarding the respective user. User information may be configured to support a self-description page (also referred to as a “user profile page”) for each user, which may be displayed as a web page or in other form. In some examples, user information may include a list of friends of the user. For example, social network server 4 may associate a user profile with a community of other users of the social network. Example communities include communities containing family members of the user, co-workers of the user, healthcare providers of the user, insured members in the same insurance group as the user, participants in a common activity of the user (e.g., religious organization, social organization, health club), and the like. In some examples, each user of users 6 can control the communities to which they belong, the amount of personal informal shared on their user profile page in each community, and the like. Social network server 4 may enable users 6 participating in the network (e.g., all users or only users within certain communities) to interact with each other in any number of ways, including blogging, discussion groups, email, file sharing, instant messaging, online chat, video, voice chat, and/or other techniques.

To establish a user profile for each user associated with social network server 4, user information for each user may be actively provided by the user, collected from user (e.g., from an employer or health insurance provider), provided from some other network, system or database that aggregates such data, or by any combination of the foregoing. User information may be included in a file or other data structure. Each element of user information may be one or more data fields, data records, or other type of data entry in a data structure. For example, user information stored in a profile for a user may include user identity data, such as gender, age, race, name, a status of the user or member (e.g., an online status or a non-online related status) (e.g., at work, at sleep, on vacation, etc.), a social security number, image information (such as a filename for a picture, avatar, or other image representative of the user or member), and/or other information associated with the user's identity. User identity information may also include e-mail addresses, login names and passwords.

Engagement with the social health and wellness network by a user and between users may be facilitated by social network server 4. FIG. 2 is a block diagram illustrating an arrangement of components of an example server 20, in accordance with one aspect of the present disclosure. Server 20 may correspond to social network server 4 of FIG. 1. FIG. 2 illustrates only one particular example of server 4, however, and other example configurations of server 4 may be used. Further, although server 20 is illustrated as a single server device, server 20 may be realized by a single computing device or a plurality of cooperating computing devices which, in combination, perform the functions attributed to server 20 herein.

In the example of FIG. 2, server 20 includes control unit 22, network interfaces 24, administrator interface 26, and database 28. Control unit 22 includes wellness rewards module 30, wellness encouragement module 32, wellness analytics module 34, user interface control module 36, wellness community module 38, and wellness challenges module 40. Administrator interface 26 allows an administrator 40 to configure server 20.

Control unit 22 may include any combination of hardware, software, and/or firmware for performing the functions attributed to control unit 22. For example, control unit 22 may include a computer-readable storage medium encoded with instructions for wellness rewards module 30, wellness encouragement module 32, wellness analytics module 34, user interface control module 36, wellness community module 38, and wellness challenges module 40, as well as a processor that executes the instructions. In another example, control unit 22 may include one or more microprocessors, digital signal processors (DSPs), application specific integrated circuits (ASICs), field programmable gate arrays (FPGAs), or any other equivalent integrated or discrete logic circuitry, as well as any combinations of such components. Similarly, any or all of wellness rewards module 30, wellness encouragement module 32, wellness analytics module 34, user interface control module 36, wellness community module 38, and wellness challenges module 40 may be functionally integrated.

Network interfaces 24 receive and output data over a network, such as the Internet. In one example, one of network interfaces 24 may correspond to an interface for receiving data from user devices 8. For example, one of network interfaces 24 may correspond to an interface for receiving data from a telemetry module of a user device. In any case, input interfaces of network interfaces 24 receive data including data generated by a health measurement device used by a user. Output interfaces of network interfaces 24 may, in various examples, include one or more interfaces for outputting data to user devices 8, including data in the form of a message encouraging a user to start, stop, or continue certain physical activity, data initiating a wellness challenge, data indicating that a user has earned an incentive award, or the like. In some examples, input and output network interfaces 24 may be functionally integrated, while in other examples, input and output interfaces may be separate interfaces of network interfaces 24. For example, network interfaces 24 may include one or more network interface cards (NICs) configured to communicate over, for example, Ethernet, transmission control protocol (TCP), Internet protocol (IP), asynchronous transfer mode (ATM), or other network communication protocols. In some examples, server 4 may include a plurality of either or both input and output interfaces.

Wellness rewards module 30 executing on control unit 22 may receive data indicative of the health and wellness of a user, e.g., from user devices 8. In some examples, the data may be received from a health measurement device with which a user interacts. The health measurement device may measure a physical activity level of a user, an emotional state of a user, a health state of a user, or the like. For example, the health measurement device may be a device carried by a user that measures the type, magnitude, and/or duration of physical activity performed by the user. The health measurement device may measure and transmit the physical activity data to wellness rewards module 30 automatically, e.g., upon being connected to network 10. Alternatively, a user may interact with the health measurement device, e.g., to activate or deactivate the operation of the health measurement device, indicate the type of activity being performed while the health measurement device monitors the magnitude and duration of the physical activity, or the like. Wellness rewards module 30 may receive data from the health measurement device indicative of a monitored or measured condition of a user and/or any user input provided to the health measurement device.

Responsive to receiving the data, wellness rewards module 30 may determine a wellness reward for the user. The wellness reward may be in the form of points, a grade, or other metric that rates the action of the user. Further, different wellness rewards may be stored for different user actions. Wellness rewards module 30 may determine a number of wellness rewards to issue to the user based on the received data and maintain a cumulative number of wellness rewards earned by the user, e.g., in database 28.

Wellness rewards module 30 may receive other types of data to evaluate and determine a wellness reward for a user beyond data received from a health measurement device. For example, wellness rewards module 30 may receive data indicating that a user has engaged with the social network server via server 20 and issue the user different wellness rewards based on the type and/or amount of interaction with the social network. In different examples, wellness rewards module 30 may determine that a user has logged into their account associated with the social health and wellness network, determine that a user has posted a message or sent a message to another user of the network, determine that the user has added or modified a health or wellness goal, or the like. Wellness rewards module 30 may issue a wellness reward for each rewardable engagement the user has with the social network. In general, issuing wellness rewards to a user for engaging with the social network can incentivize the user to continue or increase their interaction with the social network. In turn, increased interaction may increase the health and wellness benefits achievable by that particular user as well as by other users within the network.

Wellness rewards module 30 may issue a reward based on activity performed in response to a challenge generated by wellness challenges module 40. Wellness challenges module 40 executing on control unit 22 may periodically issue reward challenges to a user to incentivize the user to perform certain actions. A reward challenge may be a special wellness reward (e.g., number of points, grade, etc.) that is offered to a user and that is earned by the user if the user performs a corresponding challenge action. For example, a challenge may require that a user engage with the social network a certain number of times, perform a certain type, magnitude, and/or duration of physical activity, add or modify settings or preferences associated with a user profile, engage with other users, or the like. By completing the challenge action, the user can earn the wellness reward associated with the challenge.

Wellness challenges module 40 may interact with other modules on control unit 22 and may control various aspects of reward challenges issued by server 20. For example, wellness challenges module 40 may determine when a reward challenge is issued, how the reward challenge is issued, and the type or types of reward challenges issues. Wellness challenge module 40 may receive data, e.g., from user devices 8 or another computing device communicatively coupled to server 20, indicating a user's willingness to participate in an issued reward challenge by registering for the challenge. Wellness challenge module 40 may also receive data indicative of a user's progress toward meeting the requirements set out in the reward challenge to earn a wellness reward.

Wellness challenges module 40 may issue a reward challenge in response to user input (e.g., from administrator 40) establishing the challenge activity and the corresponding wellness reward. Alternatively, wellness analytics module 34 may analyze a user's wellness behavior (as described in greater detail below) and cause wellness challenges module 40 to issue the reward challenge so as to incentivize a user to start, stop, or continue certain behavior. Wellness challenges module 40 may issue a reward challenge by sending a message to a user device (e.g., via network interfaces 24), displaying the reward challenge to the user (e.g., via user interface control module 36) so that the user sees the challenge when they access the social network, or the like. A reward challenge may be issued for a particular user, for a group of users (e.g., a community of users), or for all users of the social network.

For example, wellness challenges module 40 may issue a reward challenge to all users of the social health and wellness network or one or more subgroups of the network. As one example, wellness challenges module 40 may issue a reward challenge for all users of the social health and wellness network that belong to a specific business organization or a particular business unit within that business organization. As another example, wellness challenges module 40 may issue a reward challenge for all social health and wellness network users that belong to a particular fitness club or only those users associated with a particular personal trainer (e.g., exercise group leader).

Wellness challenges module 40 may separate different users of the social health and wellness network into different reward challenge brackets based on activity levels and/or health states of the users, e.g., as determined from data received from user devices 8. For example, wellness challenges module 40 may issue one reward challenge to a group of users exhibiting a certain threshold activity level and/or health state and a different reward challenge to a different group of users exhibiting a different threshold activity level and/or health state. In addition to or in lieu of issuing different reward challenges to different users, wellness challenges module 40 may prohibit a user from registering for a challenge unless they meet or exceed a threshold activity level and/or health state.

For example, if a challenge requires a user to compete in a race to earn a wellness reward, wellness challenges module 40 may prohibit a user that walks on average 3000 steps per day from registering in the same challenge wave as a user that walks an average of 10,000 steps per day. Instead, wellness challenges module 40 may separate the different users into different brackets levels for the challenge, only allowing a user to register for that bracket level which they are qualified. Wellness challenges module 40 may determine suitable bracket levels and which level a particular user belongs, e.g., based on health and wellness data received from user devices 8. Although wellness challenges module 40 may limit participation in a reward challenge to certain users, in other examples, wellness challenges module may issue a reward challenge that all users of the network can register to participate in.

Wellness challenges module 40 may issue a reward challenge that is an individual challenge, requiring only individual action to complete. Additionally or alternatively, wellness challenges module 40 may issue a reward challenge that is a group challenge, requiring multiple individuals to complete actions before the challenge is complete and wellness reward earned. For example, wellness challenges module 40 may issue a group challenge requiring an entire group of users to reach a particular milestone before the group earns the corresponding wellness reward. Wellness challenges module 40 can issue reward challenges that users can join for free (e.g., without monetary compensation) and can also issue reward challenges that require users to pay to register and join.

After wellness challenges module 40 issues the reward challenge, wellness rewards module 30 may receive data indicating a user's progress toward completing the challenge. Wellness rewards module 30 may use the data to determine if the user has completed the challenge and, if so, issue the corresponding wellness reward to the user. For example, wellness rewards module 30 may issue a wellness reward by increasing a cumulative number of wellness rewards stored for the user, e.g., in database 28.

Wellness rewards module 30 may determine whether a user has accumulated enough wellness rewards to earn an incentive award. An incentive award can be a physical item (e.g., shirts, gift certificates, physical fitness supplies), a virtual item (e.g., increased status within the social network community, digital content such as copyrighted movies, books, music), a tax free employer monetary contribution to a specialized benefit account, or the like, which incentivizes the user to engage in conduct that will improve their health and wellbeing and that recognizes the user's compliance with a program to improve their health and wellbeing. For example, upon determining a wellness reward earned by a user for a new action and incrementing the cumulative wellness rewards earned by the user, wellness rewards module 30 may compare the cumulative wellness rewards earned by the user to one or more stored thresholds. The stored thresholds may set wellness reward levels required to obtain an incentive award. For example, a certain number of wellness rewards may be required to obtain a first incentive award while a different number of wellness rewards may be required to obtain a second incentive award different than the first wellness award. The user may preselect a particular incentive award that the user is working to earn.

Wellness rewards module 30 may compare the cumulative number of wellness rewards earned by the user to a wellness reward level required to obtain an incentive award. For example, wellness rewards module 30 may determine if the cumulative number of wellness rewards earned by the user is greater than (greater than or equal to) the wellness reward level required to obtain the incentive award. If wellness rewards module 30 determines that the user has obtained enough wellness rewards to earn the incentive award, wellness rewards module 30 may issue a notification to the user that the user has earned the incentive award. For example, wellness rewards module 30 may send a message to a user device (e.g., via network interfaces 24) carried by the user, display a message to the user (e.g., via user interface control module 36) so that the user sees the message when they access the social network, or the like. Wellness rewards module 30 may further reset a cumulative number of wellness rewards stored for the user, e.g., in database 28.

In some examples, wellness rewards module 30 determines whether a user has accumulated enough wellness rewards to earn an incentive award in substantially real-time as the user is accumulating the wellness rewards. Upon determining that the user has obtained enough wellness rewards to earn the incentive award in such an example, wellness rewards module 30 may issue a notification to the user in substantially real time that the user has earned the incentive award. For example, wellness rewards module 30 may receive data from a user device (e.g., a health measurement device) in substantially real-time as the device is generating the data (e.g., as a user is measuring their health state and/or performing a certain physical activity). In response to receiving the data, wellness rewards module 30 may determine a wellness reward earned by a user based on the data, update the cumulative wellness rewards stored for the user, and compare the cumulative number of wellness rewards stored for the user to a wellness reward level required to obtain an incentive award. Wellness rewards module 30 may further issue a notification in substantially real-time to the user upon determining that the user has earned an incentive award. For example, wellness rewards module 30 may issue the notification to the user less than 1 hour after the user has engaged in conduct earning the marginal wellness reward needed to achieve the incentive award such as, e.g., less than 30 minutes, less than 10 minutes, less than 5 minutes, less than 1 minute, or less than 30 seconds. In some examples, wellness rewards module 30 issues the notification while the user is still engaging in the conduct that earned the marginal wellness reward needed to achieve the incentive award. The user may receive the notification (e.g., via a portable user device) in substantially real-time, which may reinforce the rewarded behavior of the user and cause the user to continue their immediate beneficial activity.

A notification issued by wellness rewards module 30 concerning an earned incentive award can take a number of different forms. In the example of a physical incentive award, the notification may indicate that the award has been earned, that the award is being processed, that the award is being shipped, that the award has been shipped, or the like. In the example of a digital incentive award, the notification may include any of the foregoing notifications but may also or instead include delivery of the digital incentive award itself. For example, the notification may deliver digital content (e.g., the content itself or authorization to access the content) to a user (e.g., a user device). In such an example, the user may receive the award and be able to use the award in substantially real-time with earning the award.

In some examples, a user can select (e.g., via user interface control module 36) an incentive award to be earned upon accumulation of additional wellness rewards from a menu of various incentive awards. The user can select the incentive award prior to accumulating the requisite number of wellness rewards necessary to earn the incentive award. Upon subsequently earning the amount of wellness rewards necessary to earn the pre-selected incentive award, wellness rewards module 30 can issue a notification indicating that the pre-selected incentive award has been earned. In some applications, a user can select and store a queue of multiple awards identifying both the particular incentive awards selected to be earned and the sequential order in which incentive awards are to be earned. The user may change the prioritized order of incentive awards in the queue. Upon earning the amount of wellness rewards necessary to earn the first incentive award in the queue, wellness rewards module 30 can issue a notification indicating that the incentive award has been earned. Wellness rewards module 30 can issue subsequent notifications indicating that subsequent incentive awards in the queue have been earned, as the user accumulates the requisite amount of wellness rewards necessary to earn those incentive awards.

In another example, wellness rewards module 30 may function to monitor and validate user behavior for distribution of monetary funds associated with the behavior. One example of such a monetary fund is a certain amount of money made available to a user by an employer, insurer, or other party that the user can only receive if the user engages in a certain type of predefined behavior. Another example of such a monetary fund is a certain amount of money that is authorized by government regulation to be placed in a tax-free account (e.g., similar to a health savings account) but which the user can only withdraw tax-free if he engages in a certain type of predefined behavior. The requirements for obtaining the monetary funds may or may not be stored, e.g., in database 28, and accessible to server 20. In either case, wellness rewards module 30 may receive data from a user device (e.g., a health measurement device) and may store the data in processed or unprocessed form, e.g., in database 28, for validating a user's compliance with requirements for earning the monetary funds. In instances in which the user device (e.g., health measurement device) automatically generates and transmits the data, the network environment may help ensure that a user has in fact complied with any requirements necessary to obtain the monetary funds. In some examples, wellness rewards module 30 determines if the user has met the requirements for receiving the monetary funds and, if so, issues forms, an authorization code, a monetary transfer, or otherwise provides user access to the monetary funds.

Wellness encouragement module 32 may output encouragement messages to social network users to encourage each user to start, stop, or continue certain conduct. Wellness encouragement module 32 may issue an encouragement message to one user in response to receiving the message from another user of the network. For example, one user may view another user's profile and wish to send the other user a message about a change in their health and wellness status, their progress on achieving an incentive award, or their other health and wellness behavior. Wellness encouragement module 32 may receive a message composed by one user and issue the message to another network user. Wellness encouragement module 32 may issue the message in the form of a post to a user interface associated with the user's profile on the network, an e-mail message to the user's e-mail account, a text message to a portable device of the user, or the like.

In another example, wellness analytics module 34 may analyze a user's wellness behavior (as described in greater detail below) and cause wellness encouragement module 32 to issue a message to the user based on the analysis. For example, wellness analytics module 34 may receive data from a user device (e.g., a health measurement device) and determine that the user is engaging in positive or negative health behavior. In response to determining that the user is engaging in positive or negative health behavior, wellness encouragement module 32 may issue a message encouraging the user to continue the behavior (e.g., “keep exercising,” “way to go,” “you've met your weight goal,” “keep going, you about to earn an incentive award”) or start a new behavior (e.g., “have you planned your exercise time for the day yet,” “it's been 3 days since you exercised,” “it's time to schedule your annual doctor's appointment.”). The user may receive the message via a user device (e.g., a mobile phone), prompting the user to continue or modify health and wellness behaviors.

Wellness analytics module 34 may analyze health and wellness data received from network users (e.g., via health measurement devices). In general, wellness analytics module 34 may perform any data analysis techniques useful to track health and wellness of the users. For example, wellness analytics module 34 may analyze the type, magnitude, and/or duration of physical activity performed by a user to determine how the user's physical activity behavior is changing over time. As another example, wellness analytics module 34 may analyze health state data determined for a user to determine how the user's health state is changing over time. Wellness analytics module 34 may also compare the user's physical activity behavior and/or health state information to other groups of individuals to benchmark the user's health and wellness. In different examples, wellness analytics module 34 may compare the user's physical activity behavior and/or health state information to other individuals in the user's social network community, other individuals in the social network at large, or society in general. For example, wellness analytics module 34 may compare indicators of the user's physical activity behavior and/or health state to average indicators of a group of other users participating in the social network and/or a group of similar individuals (e.g., similar age and gender) in society.

In one example, wellness analytics module 34 analyzes cumulative wellness reward data for a user, e.g., stored in database 28, to determine a rolling cumulative wellness reward average for the user. As indicated above, wellness rewards may be indicative of a user's physical activity level and/or engagement with the social network, and the more wellness rewards the user accumulates the more the user may be engaging in behavior that promotes a healthy and active lifestyle. Accordingly, monitoring changes in the user's total number of wellness rewards, e.g., by calculating a rolling average of the wellness rewards, may provide a good indication of changes in the user's lifestyle.

In different examples, wellness analytics module 34 may determine a rolling 2-day average, a rolling 3-day average, a rolling 5-day average, a rolling 7-day average, a rolling 10-day average, or the like for the total number of wellness rewards accumulated by the user. Wellness analytics module 34 may update the rolling average as the user earns new rewards.

User interface control module 36 may modify a user's profile in an account stored in database 28 to reflect changes to the health and wellness status of the user, challenge participation information for the user, wellness rewards and an achievement award queue for the user, and the like. The user's profile may be displayed to the user by one of user devices 8. The user's updated profile may be viewed by the user's friends, family members, co-workers, or the like, with whom the user is connected via the social network. In some examples, user interface control module 36 modifies the user profile to reflect the health and wellbeing status of the user, and sends the modified user profile from server 20 to at least one device associated with at least one of the contacts of the user or the user, such that the modified user profile is displayable by the device receiving the modified user profile.

Example profile information that user interface control module 36 may modify for a user includes statistics related to the user's health and wellbeing (e.g., as determined by wellness analytics module 34), a cumulative number of wellness rewards currently earned by the user, a total number of wellness rewards needed for the user to earn an incentive award, the incentive award the user is working to earn, and the like. In general, user interface control module 36 can modify profile information for a user profile to reflect any determinations made or information generated by wellness rewards module 30, wellness encouragement module 32, and/or wellness analytics module 34.

As mentioned above, users of the social health and wellness network may be associated with a community or communities of other users of the social network. Example communities include communities containing family members of the user, co-workers of the user, healthcare providers of the user, insured members in the same insurance group as the user, participants in a common activity of the user (e.g., religious organization, social organization, health club), and the like. Wellness community module 38 can control association of users, including user profiles generated and/or stored by user interface control module 36, into different communities. Wellness community module 38 can also control sharing of user information within a user's community and across user communities.

In response to receiving user input (e.g., from user device 8), wellness community module 38 executing on control unit 22 may associate a particular user profile with a subgroup of other user profiles belonging to the social health and wellness network. A subgroup may be an organization itself (e.g., employer, health club) within the overall social health and wellness network or a unit of users within an organization (e.g., group of employees, group of health club members). Each subgroup may or may not be hierarchical in nature.

In operation, wellness community module 38 may interact with user interface control module 36 to receive user profile information for each user in a subgroup. The profile information may contain user selections concerning what information can be shared within the network and with whom the information can be shared. Wellness community module 38 may push information to other users within the network based on these selections. For example, wellness community module 38 may control dissemination of information concerning a particular user's health and wellness achievement(s) to other members within a subgroup based on information control selections associated with that particular user's profile. For example if user “A” and user “B” were members of a given subgroup and both member's user profiles allowed other members of their subgroups to view their health and wellness related achievements (e.g., earned wellness rewards), then user “A” could view the achievements of user “B,” e.g., as the achievements are accumulated.

In some examples, wellness community module 38 can filter specific achievements from users of the subgroup for viewing by other users associated with that subgroup. For example, wellness community module 38 may filter the achievements/progress of participants in a wellness challenge issued by wellness challenge module 40 to be viewed by other members in the subgroup which are also participating in the challenge. This filtered view of subgroup member achievements may allow subgroup members to message one another in order to earn more wellness rewards and offer messages of encouragement to other members of the challenge. Such behavior may also be available to unfiltered views of subgroup member achievements.

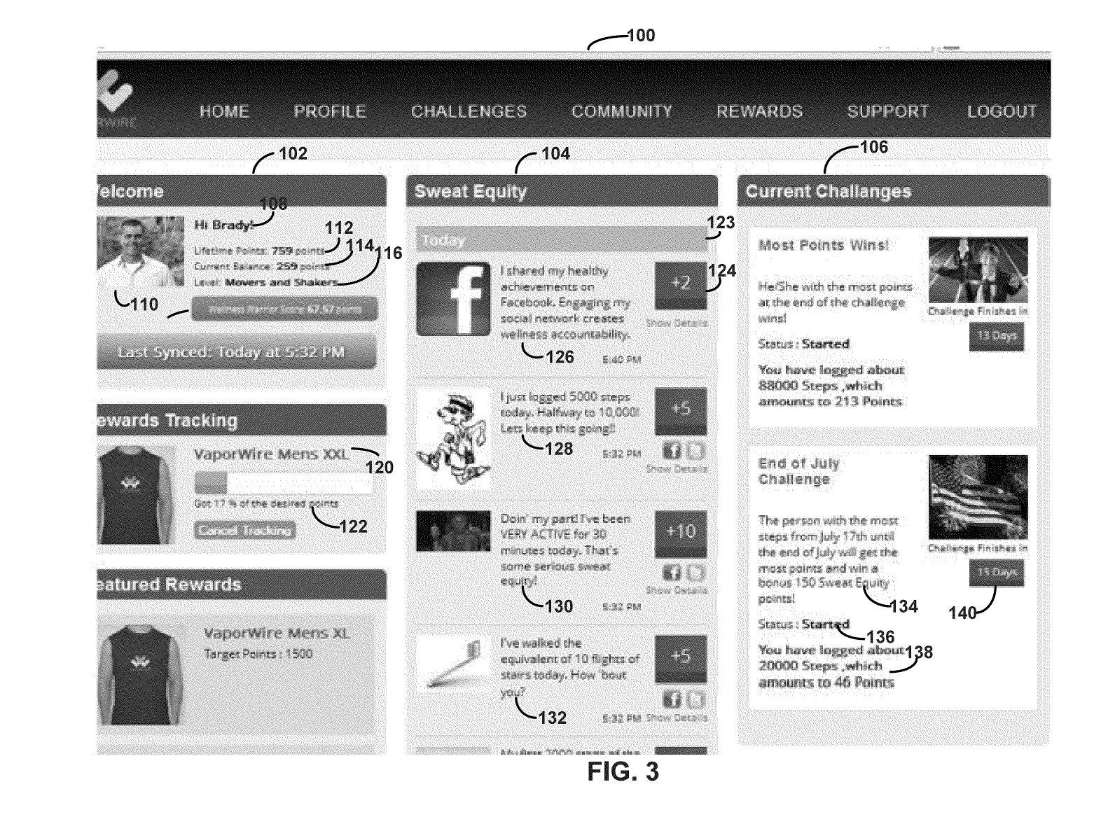

FIG. 3 illustrates an example user interface 100 that may be presented to a user by interface control module 36. User interface 100 is a display that a user might see when logged in to an online application connected to the social health and wellness network. The example display includes a user summary profile 102, a user health and wellness tracking summary 104, and an activity challenge summary 106. User summary profile 102 displays a user name 108, a user photograph 110, a cumulative number of wellness rewards 112 earned by the user over the user's entire time participating on the network, a current cumulative number of wellness rewards 114 earned by the user toward achieving the next incentive award, and a health and wellness status level 116. User summary profile 102 also displays a 7-day rolling average number of wellness rewards accumulated by user 118, an incentive award selected by the user and that the user is working to earn 120, and a progress indicator 122 indicating the user's progress toward earning the incentive award.

User health and wellness tracking summary 104 identifies activities engaged in by the user over a period of time 123 to better the user's health and wellness. User health and wellness tracking summary 104 also identifies wellness rewards 124 associated with each activity. The example activities include user engagement activities such as, e.g., sharing information about the user's health and wellness achievements 126, and physical activities such as, e.g., jogging 128, being active 130, and taking the stairs instead of the elevator 132. The activities may be entered by the user or received from a health measurement device employed by the user.

Activity challenge summary 106 identifies special challenges that the user can complete to earn extra wellness rewards. Activity challenge summary 106 includes a description 134 of the challenge, an indication 136 whether the user is participating in the challenge, a status 138 of the user's progress toward the challenge goal, and a deadline 140 when the challenge ends.

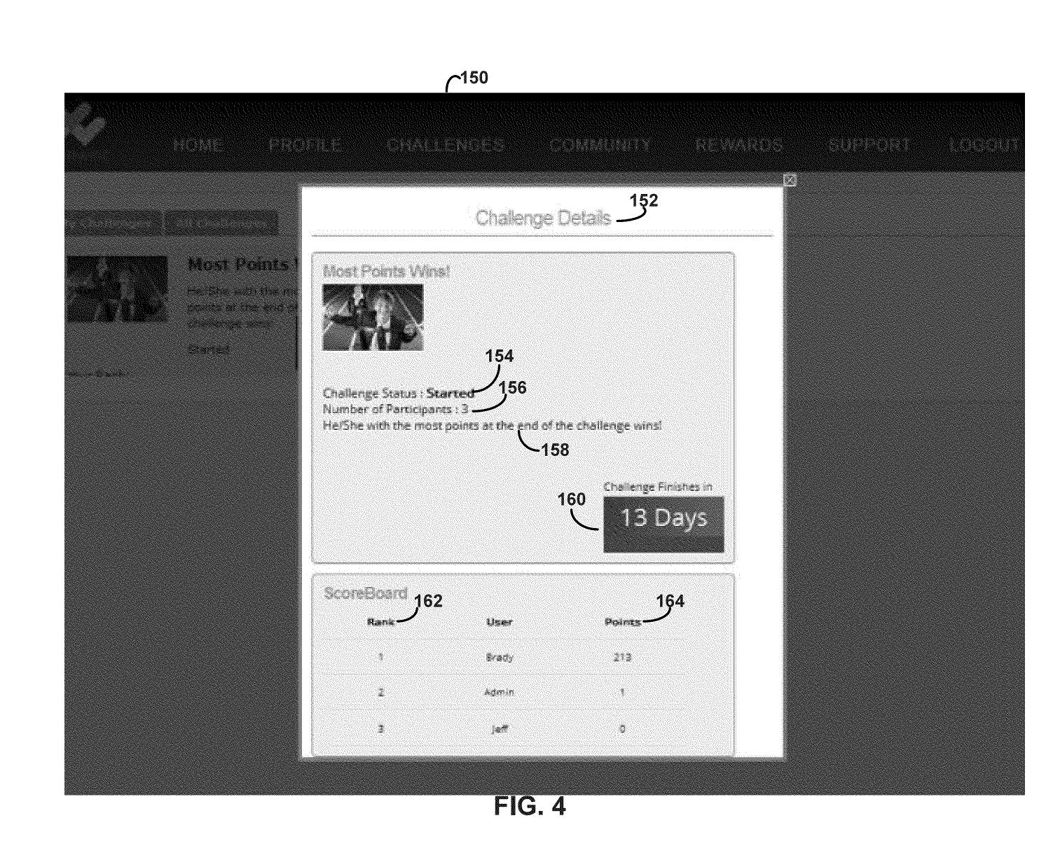

A user may select various regions of user interface 100, and user interface control module 36 may present the user consistent, follow-on user interfaces related to the region of user interface 100 selected by the user. FIG. 4 illustrates an example user interface 150 that may be presented to a user by interface control module 36 upon selecting a particular challenge in activity challenge summary 106. User interface 150 displays challenge details 152 about the ongoing challenge such as, e.g., a status 154 of the challenge, a number of participants 156 in the challenge, details 158 on winning the challenge, and a deadline 160 when the challenge ends. Challenge details 152 also indicate different participants rank 162 in the challenge and a wellness reward 164 earned by each participant participating in the challenge to date.

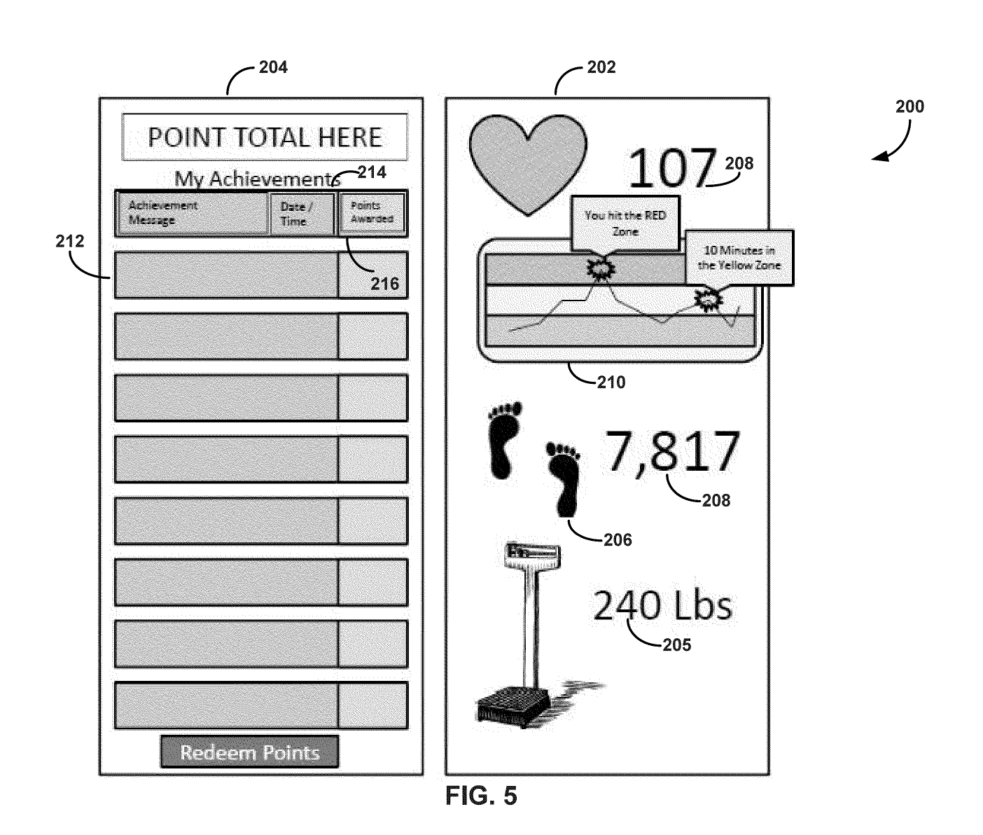

FIG. 5 illustrates another example user interface 200 that may be presented to a user by interface control module 36. In this example, user interface 200 includes health summary information 202 and wellness reward summary information 204 for a particular user. Health summary information 202 includes information on a health status 205 of the user and on a type 206, magnitude 208, and duration 210 of physical activity undertaken by the user in a given period of time. Wellness reward summary information 204 includes a listing of rewardable achievements 212 accomplished by the user, a time stamp 214 when the achievements were accomplished, and the magnitude of the wellness reward 216 associated with each achievement.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or with in separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in a computer-readable medium, such as a non-transitory computer-readable storage medium, containing instructions. Instructions embedded or encoded in a computer-readable medium, including a computer-readable storage medium, may cause one or more programmable processors, or other processors, to implement one or more of the techniques described herein, such as when instructions included or encoded in the computer-readable medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may comprise one or more computer-readable storage media.

Various embodiments of this disclosure have been described. These and other embodiments are within the scope of the following claims.

1. A method comprising:
receiving, at a server device, data related to a health of a user, the data being generated by a health measurement device configured to transmit the data over a network to the server device;
responsive to receiving the data related to the health of the user, determining, by the server device, a wellness reward for the user, wherein different wellness rewards are associated with different data received from the user; and
modifying, by the server device, a user profile associated with the user to reflect the wellness reward.

2. The method of claim 1, wherein the health measurement device is configured to measure at least one of an activity level and a health state of the user.

3. The method of claim 2, wherein the health measurement device is at least one of a pedometer, a heart rate monitor, a scale, a blood pressure measurement device, and a blood glucose measurement device.

4. The method of claim 1, further comprising comparing, by the server device, a cumulative number of wellness rewards obtained by the user with a wellness reward level required to obtain an incentive award and, if the cumulative number of wellness rewards is equal to or greater than the wellness reward level, sending a notification from the server device to a user device that the user has earned the incentive award.

5. The method of claim 4, further comprising receiving and storing a queue of multiple incentive awards to be earned and a sequential order in which the multiple incentive awards are to be earned, wherein sending the notification from the server device to the user device that the user has earned the incentive award comprises sending a notification that the user has earned a next incentive award in the queue.

6. The method of claim 4, wherein the wellness reward is a number of points and different numbers of points are associated with different activities performed by the user.

7. The method of claim 6, wherein the wellness reward level is a number of points required to obtain an incentive award.

8. The method of claim 6, wherein different incentive awards are associated with different wellness rewards levels, and further comprising receiving, by the server device, a user selection of a particular incentive award the user is working towards obtaining.

9. The method of claim 4, wherein the wellness reward includes one of a physical item and copyrighted digital content.

10. The method of claim 4, wherein the server device receives the data being generated by the health measurement device in substantially real-time, and sending the notification comprises sending the notification in substantially real-time with determining that the user earned the cumulative number of wellness rewards equal to or greater than the wellness reward level required to earn the incentive award.

11. The method of claim 10, wherein sending the notification in substantially real-time comprises sending the notification less than 5 minutes after determining that the user earned the cumulative number of wellness rewards equal to or greater than the wellness reward level required to earn the incentive award.

12. The method of claim 4, wherein sending the notification comprises sending a notification that a physical item, digital content, or tax-free monetary funds are in the process of being delivered to the user.

13. The method of claim 1, further comprising issuing, by the server device, an encouragement for the user to engage in healthy conduct, wherein issuing the encouragement comprises at least one of sending the encouragement to a user device carried by the user or associating the encouragement with the user profile so that the user receives the encouragement when viewing the profile.

14. The method of claim 13, wherein in the encouragement is a message instructing the user to start, stop, or continue engaging in certain conduct.

15. The method of claim 14, wherein the encouragement is generated by a different user associated with the server device, and further comprising receiving, by the server device, the encouragement from the different user.

16. The method of claim 13, further comprising analyzing, by the server device, the data received from the health measurement device and issuing the encouragement in response to the analysis.

17. The method of claim 1, further comprising providing, by the server device, access to tax-advantage money.

18. The method of claim 1, further comprising issuing, by the server device, a reward challenge that, if completed, earns the user wellness rewards, wherein the reward challenge comprises one of a challenge that separates different users into different challenges or challenge brackets based on activity levels of the different users, a challenge that is open to all users to join, and a challenge that is open to only members of a certain subgroup or subgroups to join.

19. The method of claim 1, further comprising organizing, by the server device, different users into different social network communities based on bio-demographic information of the different users, wherein an individual user can opt in or out of a particular social network community to which are given access.

20. A non-transitory computer readable medium comprising instructions that cause a processor to:
receive data related to a health of a user, the data being generated by a health measurement device configured to transmit the data over a network;
responsive to receiving the data related to the health of the user, determine a wellness reward for the user, wherein different wellness rewards are associated with different data received from the user; and
modify a user profile associated with the user to reflect the wellness reward.

21. A system comprising:
an interface of a server device that receives data related to a health of a user, the data being generated by a health measurement device configured to transmit the data over a network to the interface of the server device;
a wellness rewards module that determines a wellness reward for the user based on the received data; and
a user interface control module that modifies a user profile associated with the user to reflect the wellness reward.
